(19) United States
(12) Patent Application Publication
(10) Pub. No.: US 2026/0232616 A1
(43) Pub. Date: Aug. 13, 2026

(54) USE OF SIVELESTAT IN ANALGESIA

(71) Applicant: SHANGHAI HUILUN PHARMACEUTICAL CO., LTD., Shanghai (CN)

(72) Inventor: Wenhua Li, Shanghai (CN)

(21) Appl. No.: 19/471,721

(22) PCT Filed: Apr. 2, 2024
(86) PCT No.: PCT/CN2024/085416

§ 371 Date: Oct. 2, 2025

(30) Foreign Application Priority Data
Apr. 4, 2023 (CN) 202310356630.1

(51) Int. Cl. A61K 31/222; A61K 31/4468; A61K 31/451; A61K 31/4535; A61K 31/485; A61P 25/04; A61P 29/00

(52) U.S. Cl. CPC A61K 31/222; A61K 31/4468; A61K 31/451; A61K 31/4535; A61K 31/485; A61P 25/04; A61P 29/00

(57) ABSTRACT

The present invention relates to a use of sivelestat in analgesia, and in particular to a use of sivelestat or a salt thereof alone or in combination with an opioid in the preparation of a drug for treating or relieving pain, especially postoperative pain or post-traumatic pain. Sivelestat has a long-lasting analgesic effect in the treatment of postoperative pain or post-traumatic pain, and can generate a significant synergistic effect when used in combination with the opioid. The combination of sivelestat and the opioid can significantly reduce the dosage of the opioid, and can further prolong the analgesia time.

### TECHNICAL FIELD

The present disclosure belongs to the field of pharmaceutical technology, and relates in particular to use of sivelestat or a salt thereof, either alone or in combination with an opioid drug, in preparation of a drug for treating or alleviating pain.

### BACKGROUND

The International Association for the Study of Pain (IASP) has recently defined pain as an unpleasant sensory and emotional experience associated with, or resembling that associated with, actual or potential tissue damage. Based on its duration, pain can be classified as either acute pain or chronic pain. Acute pain is nociceptive pain typically lasting less than 1 month. It is often related to surgical trauma, tissue damage, or some disease states, and naturally resolves as the trauma, damage, or disease heals. Chronic pain, generally defined as pain lasting for 3 months or more, is often associated with nerve injury or inflammation, highly complex in pathogenesis, and difficult to treat, and may persist long after the primary disease or tissue damage has healed.

Postoperative pain is acute pain that occurs immediately after surgery, including both somatic pain and visceral pain, and usually lasts no more than 3 to 7 days. Postoperative pain is nociceptive pain. If not adequately controlled in its initial state, postoperative pain may develop into chronic pain (chronic post-surgical pain, CPSP) or transform into neuropathic pain or mixed pain. Severe postoperative pain often triggers a series of stress responses, which increases the incidence of complications such as cardiovascular and respiratory complications or thromboembolism, and adversely affects patients' immune function, digestive system function, endocrine metabolic function, and the like, significantly impairing patients' quality of life after surgery.

Opioid drugs are commonly used analgesics in clinical practice. They primarily realize analgesic effects by acting on opioid receptors, offering potent analgesic effect, making them irreplaceable in clinical practice. The commonly used opioid analgesics in clinical practice include morphine, tramadol, fentanyl, and oxycodone. However, the opioid drugs often cause systemic side effects and adverse reactions such as nausea, vomiting, constipation, excessive sedation, and respiratory depression. Furthermore, the opioid drugs have a short duration of analgesia, and their repeated clinical use carries side effects such as addiction and tolerance, which significantly limit their application. Therefore, it is of significant benefit to patients undergoing surgery if the dosage of the opioid drugs is reduced and their analgesic duration is extended while ensuring satisfactory analgesic effect, thereby minimizing adverse effects and side effects.

Sivelestat is a selective neutrophil elastase (NE) inhibitor. It was the first drug approved globally for the treatment of acute lung injury accompanied by systemic inflammatory response syndrome. The daily dosage for this disease is 4.8 mg/kg/d (continuous intravenous administration at 0.2 mg/kg/h for 24 h). Clinical studies have confirmed that sivelestat sodium has no significant adverse reactions or side effects when administered to humans. Due to its highly selective inhibitory effect on NE, sivelestat is expected to be used in the treatment of various diseases.

Patent WO2016050835 A2 describes the use of sivelestat for treating neuropathic pain or chronic pain conditions with neuropathic pain. Neuropathic pain refers to a pain syndrome directly caused by a lesion or disease of the somatosensory nervous system. Chronic neuropathic pain is chronic pain caused by nerve injury or pathology caused by trauma, inflammation or other diseases. There are essential differences in pathogenesis and treatment regimens between neuropathic pain or chronic neuropathic pain and nociceptive pain, such as postoperative pain or posttraumatic pain.

Currently, especially for moderate to severe postoperative pain, there have been no reports on the use of neutrophil elastase inhibitors, either alone or in combination with opioid receptor agonists, for treatment. There are diverse treatment regimens and management models for postoperative pain in clinical practice. However, few single drugs can achieve effective control without significant adverse side effects. Therefore, the development of effective treatment regimens in the field of analgesia remains an important research direction.

### SUMMARY

According to a first aspect of the present disclosure, provided is use of sivelestat or a pharmaceutically acceptable salt thereof in preparation of a drug for treating or alleviating pain. The pain according to the present disclosure includes acute pain or chronic pain; the acute pain includes posttraumatic pain and postoperative pain; and the chronic pain includes inflammatory pain.

As an embodiment of the present disclosure, provided is use of sivelestat or a pharmaceutically acceptable salt thereof in preparation of a drug for treating or alleviating posttraumatic pain or postoperative pain.

As an embodiment of the present disclosure, provided is use of sivelestat sodium in preparation of a drug for treating or alleviating posttraumatic pain or postoperative pain.

As an embodiment of the present disclosure, the dose of the sivelestat or the pharmaceutically acceptable salt thereof is 1.0-500 mg/kg/d.

As an embodiment of the present disclosure, the sivelestat or the pharmaceutically acceptable salt thereof is an independent formulation including 50-500 mg of the sivelestat or the pharmaceutically acceptable salt thereof.

According to a second aspect of the present disclosure, provided is use of sivelestat or a pharmaceutically acceptable salt thereof in combination with an opioid drug in preparation of a drug for treating or alleviating pain. The pain according to the present disclosure includes acute pain or chronic pain; the acute pain includes posttraumatic pain and postoperative pain; and the chronic pain includes neuropathic pain and inflammatory pain.

As an embodiment of the present disclosure, provided is use of sivelestat or a pharmaceutically acceptable salt thereof in combination with an opioid drug in preparation of a drug for treating or alleviating posttraumatic pain or postoperative pain.

The opioid drug is a full agonist, partial/mixed agonist, or antagonist of μ-, δ-, κ-, and σ-opioid receptors; a preferred opioid drug is a full agonist of μ-opioid receptor; specific full agonists of μ-opioid receptor include morphine, fentanyl, remifentanil, sufentanil, pethidine, oxycodone, and hydromorphone; and preferred full agonists of μ-opioid receptor include morphine, fentanyl, remifentanil, sufentanil, and pethidine.

As an embodiment of the present disclosure, the dose of the sivelestat or the pharmaceutically acceptable salt thereof is 1.0-500 mg/kg/d.

As an embodiment of the present disclosure, the sivelestat or the pharmaceutically acceptable salt thereof is an independent formulation including 50-500 mg of the sivelestat or the pharmaceutically acceptable salt thereof; and the opioid drug is an independent formulation including 0.01-100 mg of the opioid drug.

According to a third aspect of the present disclosure, provided is use of sivelestat or a pharmaceutically acceptable salt thereof in combination with morphine or a pharmaceutically acceptable salt thereof in preparation of a drug for treating or alleviating pain. The pain includes acute pain or chronic pain; the acute pain includes posttraumatic pain and postoperative pain; and the chronic pain includes neuropathic pain and inflammatory pain.

As an embodiment of the present disclosure, provided is use of sivelestat sodium in combination with morphine or a pharmaceutically acceptable salt thereof in preparation of a drug for treating or alleviating posttraumatic pain or postoperative pain.

As an embodiment of the present disclosure, the dose of the sivelestat or the pharmaceutically acceptable salt thereof is 1.0-500 mg/kg/d.

As an embodiment of the present disclosure, the sivelestat or the pharmaceutically acceptable salt thereof is an independent formulation including 50-500 mg of the sivelestat or the pharmaceutically acceptable salt thereof; and the morphine or the pharmaceutically acceptable salt thereof is an independent formulation including 0.01-100 mg of the morphine or the pharmaceutically acceptable salt thereof.

As an embodiment of the present disclosure, provided is use of sivelestat sodium in combination with morphine or a pharmaceutically acceptable salt thereof in preparation of a drug for treating or alleviating posttraumatic pain or postoperative pain; the sivelestat sodium is an independent formulation including 50-500 mg of the sivelestat sodium; and the morphine or the pharmaceutically acceptable salt thereof is an independent formulation including 0.01-100 mg of the morphine or the pharmaceutically acceptable salt thereof.

According to a fourth aspect of the present disclosure, provided is a combination product including the following components:
(1) sivelestat or a pharmaceutically acceptable salt thereof; and
(2) an opioid drug.

As an embodiment of the present disclosure, in the combination product, the dose of the sivelestat or the pharmaceutically acceptable salt thereof is 1.0-500 mg/kg/d.

As an embodiment of the present disclosure, in the combination product, the sivelestat or the pharmaceutically acceptable salt thereof is an independent formulation including 50-500 mg of the sivelestat or the pharmaceutically acceptable salt thereof; and in the combination product, the opioid drug is an independent formulation including 0.01-100 mg of the opioid drug.

The opioid drug is a full agonist, partial/mixed agonist, or antagonist of μ-, δ-, κ-, and σ-opioid receptors; a preferred opioid drug is a full agonist of μ-opioid receptor; specific full agonists of μ-opioid receptor include morphine, fentanyl, remifentanil, sufentanil, pethidine, oxycodone, and hydromorphone; and preferred full agonists of μ-opioid receptor include morphine, fentanyl, remifentanil, sufentanil, and pethidine.

According to a fifth aspect of the present disclosure, provided is a combination product, including the following components:
(1) sivelestat sodium or a pharmaceutically acceptable salt thereof; and
(2) morphine or fentanyl.

As an embodiment of the present disclosure, in the combination product, the dose of the sivelestat sodium is 1.0-500 mg/kg/d.

As an embodiment of the present disclosure, in the combination product, the sivelestat sodium is an independent formulation including 50-500 mg of the sivelestat sodium; and in the combination product, the morphine, fentanyl, or the pharmaceutically acceptable salt thereof is an independent formulation including 0.01-100 mg of the morphine, fentanyl, or the pharmaceutically acceptable salt thereof.

According to a sixth aspect of the present disclosure, provided is use of a combination product in preparation of a drug for treating or alleviating pain, where the combination product is any combination product according to the present disclosure.

The pain includes acute pain or chronic pain; the acute pain includes posttraumatic pain and postoperative pain; and the chronic pain includes inflammatory pain.

The present disclosure further provides use of a combination product in preparation of a drug for treating or alleviating posttraumatic pain or postoperative pain, where the combination product is any combination product according to the present disclosure.

According to a seventh aspect of the present disclosure, provided is a pharmaceutical kit including any combination product according to the present disclosure.

According to an eighth aspect of the present disclosure, provided is a method for treating or alleviating pain, including administering to a patient in need thereof an effective amount of sivelestat or a pharmaceutically acceptable salt thereof, and an opioid drug. The pain according to the present disclosure includes acute pain or chronic pain; the acute pain includes posttraumatic pain and postoperative pain; and the chronic pain includes neuropathic pain and inflammatory pain.

As an embodiment of the present disclosure, provided is a method for treating or alleviating posttraumatic pain or postoperative pain, including administering to a patient in need thereof an effective amount of sivelestat or a pharmaceutically acceptable salt thereof, and an opioid drug.

As an embodiment of the present disclosure, provided is a method for treating or alleviating posttraumatic pain or postoperative pain, including administering to a patient in need thereof an effective amount of sivelestat or a pharmaceutically acceptable salt thereof, and morphine, fentanyl or a pharmaceutically acceptable salt thereof.

As an embodiment of the present disclosure, the dosage of sivelestat or a pharmaceutically acceptable salt thereof is 1.0-500 mg/kg/d; and the dosage of the opioid drug is 0.0001-100 mg/kg/d.

As an embodiment of the present disclosure, the method includes administering to a patient in need thereof an independent formulation including 50-500 mg of sivelestat or a pharmaceutically acceptable salt thereof, and an independent formulation including 0.01-100 mg of an opioid drug.

According to a ninth aspect of the present disclosure, provided is a method for treating or alleviating pain, including administering to a patient in need thereof any combination product or the pharmaceutical kit according to the present disclosure. The pain according to the present disclosure includes acute pain or chronic pain; the acute pain includes posttraumatic pain and postoperative pain; and the chronic pain includes neuropathic pain and inflammatory pain.

As an embodiment of the present disclosure, provided is a method for treating or alleviating posttraumatic pain or postoperative pain, including administering to a patient in need thereof any combination product or the pharmaceutical kit according to the present disclosure.

The sivelestat or the pharmaceutically acceptable salt thereof according to the present disclosure can be used to treat or alleviate pain, especially postoperative pain and posttraumatic pain. It has a long-lasting analgesic effect and maintains a high level of analgesic effect over an extended period.

By combining sivelestat or a pharmaceutically acceptable salt thereof with an opioid drug, especially in combination with morphine, the present disclosure has unexpectedly found that pain, especially posttraumatic or postoperative pain, can be significantly relieved or alleviated, and the two act synergistically when used in combination. The combination of sivelestat sodium with morphine according to the present disclosure can significantly reduce the dosage of the opioid drug, thereby decreasing adverse reactions caused by opioid drug use, as well as side effects such as addiction and tolerance. The present disclosure also solves the problems of short duration of analgesia of the opioid drug, which necessitates frequent repeated administration in clinical practice. The combination significantly prolongs the therapeutic duration of the opioid drug for pain and maintains a high level of analgesic effect over an extended period.

### BRIEF DESCRIPTION OF THE DRAWINGS

FIG. 1: anti-allodynic effect (MPE %) (6 h), *p<0.05, **p<0.01, ***p<0.001 vs the normal saline group, using one-way analysis of variance with Bonferroni multiple comparison test.

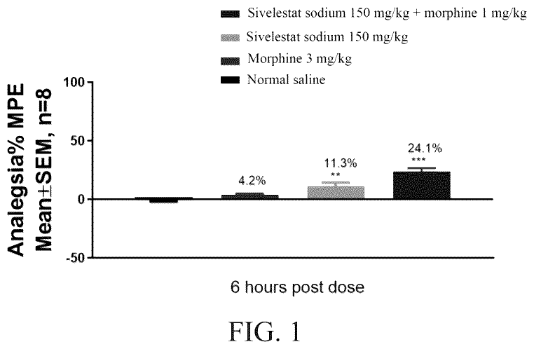

### DETAILED DESCRIPTION

### Terminology Explanation

The “sivelestat” according to the present disclosure has the following structure:

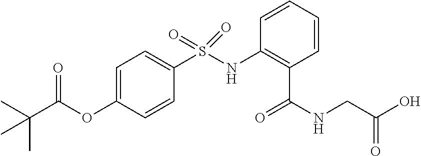 [SMILES: CC(C)(C)C(=O)Oc1ccc(S(=O)(=O)Nc2ccccc2C(=O)NCC(=O)O)cc1]

The “pharmaceutically acceptable salt” may be, but is not limited to, sodium salt, potassium salt, and calcium salt. Preferably, the pharmaceutically acceptable salt is the sodium salt. As a specific implementation of the present disclosure, the sivelestat or the pharmaceutically acceptable salt thereof is sivelestat sodium salt, also referred to as sivelestat sodium. Further, sivelestat sodium may also exist in the form of a hydrate, such as a monohydrate, dihydrate, trihydrate, or tetrahydrate. In a preferred implementation of the present disclosure, the sivelestat sodium is a sivelestat sodium tetrahydrate.

The opioid drug according to the present disclosure is selected from full agonists, partial/mixed agonists, and antagonists of μ-, δ-, κ-, and σ-opioid receptors. The full agonists include morphine, fentanyl, remifentanil, sufentanil, pethidine, etorphine, oxycodone, and hydromorphone. The partial/mixed agonists include pentazocine, nalorphine, and buprenorphine. The antagonists include naloxone and naltrexone. The full agonists of the μ-opioid receptor include morphine, fentanyl, remifentanil, sufentanil, pethidine, oxycodone, and hydromorphone. The partial/mixed agonists of the k-opioid receptor include pentazocine. Preferred opioid drugs are full agonists of opioid receptors. Preferred opioid drugs are full agonists of μ-opioid receptor. Specific full agonists of μ-opioid receptor include morphine, fentanyl, remifentanil, sufentanil, pethidine, oxycodone, and hydromorphone. Preferred full agonists of μ-opioid receptor include morphine, fentanyl, remifentanil, sufentanil, and pethidine. The opioid drug according to the present disclosure may also be administered in the form of a pharmaceutically acceptable salt thereof. It may be, for example, hydrochloride, sulfate, phosphate, citrate, tartrate, and the like. In specific embodiments of the present disclosure, the opioid drug is morphine hydrochloride or morphine sulfate. In specific embodiments of the present disclosure, the opioid drug is fentanyl citrate.

The “acute pain” according to the present disclosure primarily refers to posttraumatic pain or postoperative pain (post-op pain for short), which occurs immediately after trauma or surgery and is generally short in duration, typically resolving within 1 month. The intensity of the acute pain may be mild, moderate, or severe.

The “chronic pain” according to the present disclosure refers to pain that persists for an extended duration (e.g., exceeding 3 months) and mainly involves pain associated with various diseases such as inflammatory pain (e.g., pain resulting from rheumatoid arthritis), neuropathic pain, and mixed pain.

### Dosage Form and Dose Selection

The sivelestat or the pharmaceutically acceptable salt thereof according to the present disclosure may be adaptively adjusted according to subjects such as factors including human body weight, gender, age, and severity of pain and may be administrated via a single daily dose, multiple daily doses, or continuous administration. When calculated based on the total daily dosage, a suitable dosage of sivelestat is 0.1-500 mg/kg/d; a preferred dosage is 0.5-400 mg/kg/d; a further preferred dosage is 0.5-300 mg/kg/d; a further preferred dosage is 1-300 mg/kg/d; a further preferred dosage is 1-200 mg/kg/d; and a further preferred dosage is 1-150 mg/kg/d. In specific implementations of the present disclosure, the daily dosage of sivelestat or a salt thereof may be 1.0 mg/kg/d, 2.0 mg/kg/d, 3.0 mg/kg/d, 4.0 mg/kg/d, 4.8 mg/kg/d, 5.0 mg/kg/d, 6.0 mg/kg/d, 7.0 mg/kg/d, 8.0 mg/kg/d, 9.0 mg/kg/d, 10.0 mg/kg/d, 15.0 mg/kg/d, 20.0 mg/kg/d, 25.0 mg/kg/d, 30.0 mg/kg/d, 35.0 mg/kg/d, 40.0 mg/kg/d, 50.0 mg/kg/d, 60.0 mg/kg/d, 70.0 mg/kg/d, 80.0 mg/kg/d, 90.0 mg/kg/d, 100.0 mg/kg/d, 150.0 mg/kg/d, 200.0 mg/kg/d, 250.0 mg/kg/d, 300.0 mg/kg/d, 400.0 mg/kg/d, or 500.0 mg/kg/d.

The opioid drug of the present disclosure may be adaptively adjusted according to subjects, such as factors including human body weight, gender, age, severity of pain, and history of analgesic use, and may be administered via a single daily dose, multiple daily doses, or continuous administration. When calculated based on the total daily dosage, a suitable dosage of the opioid drug is 0.0001-100 mg/kg/d; a preferred dosage is 0.0001-50 mg/kg/d; a further preferred dosage is 0.0001-25 mg/kg/d; a further preferred dosage is 0.0005-25 mg/kg/d; a further preferred dosage is 0.001-10 mg/kg/d; and a further preferred dosage is 0.005-10 mg/kg/d. In specific implementations of the present disclosure, the daily dosage of the opioid drug may be 0.0001 mg/kg/d, 0.00015 mg/kg/d, 0.0002 mg/kg/d, 0.0003 mg/kg/d, 0.0004 mg/kg/d, 0.0005 mg/kg/d, 0.0006 mg/kg/d, 0.0007 mg/kg/d, 0.0008 mg/kg/d, 0.0009 mg/kg/d, 0.001 mg/kg/d, 0.002 mg/kg/d, 0.004 mg/kg/d, 0.005 mg/kg/d, 0.006 mg/kg/d, 0.008 mg/kg/d, 0.01 mg/kg/d, 0.05 mg/kg/d, 0.1 mg/kg/d, 0.2 mg/kg/d, 0.3 mg/kg/d, 0.4 mg/kg/d, 0.5 mg/kg/d, 0.6 mg/kg/d, 0.7 mg/kg/d, 0.8 mg/kg/d, 0.9 mg/kg/d, 1.0 mg/kg/d, 1.5 mg/kg/d, 2.0 mg/kg/d, 3.0 mg/kg/d, 4.0 mg/kg/d, 5.0 mg/kg/d, 6.0 mg/kg/d, 7.0 mg/kg/d, 8.0 mg/kg/d, 9.0 mg/kg/d, 10.0 mg/kg/d, 25.0 mg/kg/d, 50.0 mg/kg/d, 60.0 mg/kg/d, 80.0 mg/kg/d, or 100.0 mg/kg/d.

In specific embodiments of the present disclosure, when sivelestat or a pharmaceutically acceptable salt thereof is used in combination with an opioid drug, the sivelestat or the pharmaceutically acceptable salt thereof is an independent formulation including 50 mg, 60 mg, 70 mg, 80 mg, 90 mg, 100 mg, 150 mg, 200 mg, 250 mg, 300 mg 350 mg, 400 mg, 450 mg, or 500 mg of the sivelestat or the pharmaceutically acceptable salt thereof; and the opioid drug is an independent formulation including 0.01 mg, 0.02 mg, 0.04 mg, 0.05 mg, 0.075 mg, 0.1 mg, 0.25 mg, 0.3 mg, 0.5 mg, 0.8 mg, 1 mg, 2 mg, 3 mg, 4 mg, 5 mg, 8 mg, 10 mg, 15 mg, 20 mg, 25 mg, 30 mg, 35 mg, 40 mg, 45 mg, 50 mg, 60 mg, 70 mg, 80 mg, 90 mg, or 100 mg of the opioid drug.

In specific embodiments of the present disclosure, when sivelestat or a pharmaceutically acceptable salt thereof is used in combination with morphine or a pharmaceutically acceptable salt thereof, the sivelestat or the pharmaceutically acceptable salt thereof is an independent formulation including 50 mg, 60 mg, 70 mg, 80 mg, 90 mg, 100 mg, 150 mg, 200 mg, 250 mg, 300 mg, 350 mg, 400 mg, 450 mg, or 500 mg of the sivelestat or the pharmaceutically acceptable salt thereof; and the morphine or the pharmaceutically acceptable salt thereof is an independent formulation including 0.01 mg, 0.02 mg, 0.04 mg, 0.05 mg, 0.075 mg, 0.1 mg, 0.3 mg, 0.5 mg, 0.8 mg, 1 mg, 2 mg, 3 mg, 4 mg, 5 mg, 8 mg, 10 mg, 15 mg, 20 mg, 25 mg, 30 mg, 35 mg, 40 mg, 45 mg, 50 mg, 60 mg, 70 mg, 80 mg, 90 mg, or 100 mg of the morphine or the pharmaceutically acceptable salt thereof.

The sivelestat or the pharmaceutically acceptable salt thereof and the opioid drug according to the present disclosure may be administered simultaneously, sequentially, or at staggered times; before meals or after meals; and continuously or intermittently.

The sivelestat or the pharmaceutically acceptable salt thereof and the opioid drug according to the present disclosure may be administered via any conventional route, such as oral administration, injection administration, and inhalation administration.

The sivelestat or opioid drug according to the present disclosure may be combined with any pharmaceutically acceptable excipient to prepare a pharmaceutically acceptable independent formulation, including: oral solid formulations, oral liquid formulations, injections, inhalable solid formulations, inhalable liquid formulations, and sustained-release formulations. Preferred formulations are inhalable solid formulations and injections. In a specific implementation of the present disclosure, sivelestat sodium is a lyophilized formulation for injection, and the opioid drug is an injection solution.

Pharmaceutically acceptable excipients include supports, pH adjusters, preservatives, and surfactants. Examples of the supports include mannitol. Examples of the pH adjusters include phosphoric acid, sodium dihydrogen phosphate, disodium hydrogen phosphate, and sodium hydroxide.

The following animal experiments are used to further illustrate the concept of the present disclosure and should not be construed as limiting the present disclosure in any way. The animals, reagents, sivelestat sodium, and morphine used in the embodiments of the present disclosure are all commercially available.

### Embodiment 1

Evaluation of efficacy of sivelestat sodium in combination with morphine in a rat model of postoperative pain

### Animal Model and Experimental Protocol

Male SD rats weighing 200-230 g were selected, fed with standard laboratory food, and allowed free access to the food. The animals were acclimatized to the testing environment for 3 days prior to modeling. On the day of modeling, the animals were anesthetized with a combination of Zoletil 50+xylazine hydrochloride (20 mg/kg+8 mg/kg, intraperitoneal injection). A longitudinal incision approximately 1 cm in length was made on the left hind paw of the animal, 0.7-0.8 cm proximal to the heel, extending toward the toes. The skin was incised, and then flexor digitorum brevis muscle was elevated and subjected to longitudinal blunt dissection. On the first day after surgery, the baseline values of mechanical allodynia were measured in experimental rats. Based on the baseline values of the mechanical allodynia, the animals were randomly divided into 11 groups, with 8 rats in each group. The animal experimental protocol is shown in Table below.

TABLE 1
Experimental protocol

| | | Route of | | | Number |
|---|---|---|---|---|---|
| Group | | adminis- | Dosage | Volume | of |
| No. | Group | tration | (mg/kg) | (mL/kg) | animals |
| 1 | Normal saline | IP | — | — | 8 |
| 2 | Morphine | SC | 1 | 5 | 8 |
| 3 | Morphine | SC | 2 | 5 | 8 |
| 4 | Morphine | SC | 3 | 5 | 8 |
| 5 | Sivelestat sodium | IP | 100 | 5 | 8 |
| 6 | Sivelestat sodium | IP | 150 | 5 | 8 |
| 7 | Sivelestat sodium | IP | 200 | 5 | 8 |
| 8 | Sivelestat sodium | IP | 300 | 5 | 8 |
| 9 | Sivelestat sodium + | IP + SC | 100 + 1 | 5 | 8 |
| | morphine | | | | |
| 10 | Sivelestat sodium + | IP + SC | 150 + 1 | 5 | 8 |
| | morphine | | | | |
| 11 | Sivelestat sodium + | IP + SC | 200 + 1 | 5 | 8 |
| | morphine | | | | |

Note:
Route of administration: SC: subcutaneous injection; and IP: intraperitoneal injection.

### Compound Preparation

TABLE 2
Compound preparation

| | Dose | Concentration | Volume | Compound |
|---|---|---|---|---|
| Drug | (mg/kg) | (mg/mL) | (mL) | weight (mg) |
| Normal saline | — | — | — | — |
| Morphine | 1 | 0.2 | 31.84 | 6.40 |
| Morphine | 1 | 0.2 | 33.68 | 6.77 |
| Morphine | 1 | 0.2 | 33.58 | 6.75 |
| Morphine | 2 | 0.4 | 19.30 | 7.76 |
| Morphine | 3 | 0.6 | 14.31 | 8.63 |
| Sivelestat sodium | 100 | 20 | 28.746 | 574.92 |
| Sivelestat sodium | 150 | 30 | 28.894 | 866.81 |
| Sivelestat sodium | 200 | 40 | 28.892 | 1155.66 |
| Sivelestat sodium | 300 | 60 | 14.463 | 867.79 |

Morphine preparation method: accurately weigh a corresponding amount of morphine, add a corresponding volume of normal saline, and vortex.

Sivelestat sodium preparation method: accurately weigh a corresponding amount of sivelestat sodium, add an appropriate volume of normal saline containing NaOH at a concentration of 1.1 mg/mL, vortex, and adjust the pH to 7.8 using a 1 N HCl solution.

### Mechanical Allodynia Testing:

Baseline value measurement: on the first day after surgery, the baseline values of mechanical allodynia were measured in experimental rats, and according to the test results, the rats were randomly grouped.

Administration and testing: administration (a single dose) was performed on the first day after surgery, mechanical allodynia thresholds of experimental rats in each group were measured at 0.5 h, 1 h, 2 h, and 6 h after administration, and the average value was calculated. The results are shown in Table 3. The anti-allodynic effect (MPE %, MPE %=(Mechanical allodynia threshold of the test group-Mechanical allodynia threshold of the normal saline group)/(15−Mechanical allodynia threshold of the normal saline group)*100%) for each group was calculated according to the mechanical allodynia threshold. The experimental results are shown in Table 4.

TABLE 3
Mechanical allodynia threshold (PWT (g)) of each group

| | Mechanical allodynia testing (PWT (g)) | | | | |
|---|---|---|---|---|---|
| | Baseline | | | | |
| Group No. | value | 0.5 h | 1 h | 2 h | 6 h |
| Normal saline | 2.8 | 2.2 | 2.5 | 2.9 | 2.5 |
| Morphine 1 mg/kg | 2.8 | 3.7 | 3.4 | 3.3 | |
| Morphine 2 mg/kg | 2.8 | 7.1 | 8.8 | 5.8 | |
| Morphine 3 mg/kg | 2.8 | 9.9 | 13.5 | 8.1 | 3.1 |
| Sivelestat sodium 100 mg/kg | 2.9 | 5.5 | 7.8 | 8.3 | |
| Sivelestat sodium 150 mg/kg | 2.9 | 6.3 | 8.9 | 8.8 | 3.9 |
| Sivelestat sodium 200 mg/kg | 2.9 | 6.4 | 9.4 | 8.4 | |
| Sivelestat sodium 300 mg/kg | 2.9 | 7.0 | 8.6 | 9.3 | |
| Sivelestat sodium + morphine | 2.9 | 6.6 | 9.9 | 8.0 | |
| 100 mg/kg + 1 mg/kg | | | | | |
| Sivelestat sodium + morphine | 2.8 | 7.1 | 10.6 | 9.3 | 5.5 |
| 150 mg/kg + 1 mg/kg | | | | | |
| Sivelestat sodium + morphine | 2.8 | 7.0 | 11.0 | 9.7 | |
| 200 mg/kg + 1 mg/kg | | | | | |

TABLE 4
Anti-allodynic effect (MPE %) of each group

| Group No. | % MPE (anti-allodynic effect) | | | |
|---|---|---|---|---|
| Testing time point | 0.5 h | 1 h | 2 h | 6 h |
| Normal saline | 0 | 0 | 0 | 0 |
| Morphine 1 mg/kg | 11.5 | 7.3 | 3 | — |
| Morphine 2 mg/kg | 37.9** | 50.1*** | 23.9 | — |
| Morphine 3 mg/kg | 59.7*** | 88.0*** | 43.0*** | 4.2 |
| Sivelestat sodium 100 mg/kg | 25.2* | 42.4*** | 44.1*** | — |
| Sivelestat sodium 150 mg/kg | 31.4*** | 51.1*** | 48.4*** | 11.3 |
| Sivelestat sodium 200 mg/kg | 32.6*** | 55.1*** | 45.3*** | — |
| Sivelestat sodium 300 mg/kg | 37.5*** | 49*** | 52.6*** | — |
| Sivelestat sodium + morphine | 34.3*** | 59.0*** | 41.7*** | — |
| 100 mg/kg + 1 mg/kg | | | | |
| Sivelestat sodium + morphine | 37.8*** | 64.6*** | 52.9*** | 24.1*** |
| 150 mg/kg + 1 mg/kg | | | | |
| Sivelestat sodium + morphine | 37.6*** | 68.0*** | 56.1*** | — |
| 200 mg/kg + 1 mg/kg | | | | |

*p<0.05, **p<0.01, ***p<0.001 vs the normal saline group, using one-way analysis of variance with Bonferroni multiple comparison test.

As can be seen from the experimental results of the present disclosure:

(1) Sivelestat sodium can be used to treat or alleviate postoperative pain or posttraumatic pain. Its analgesic effect is long-lasting and superior to that of morphine in duration, maintaining a high level of analgesic efficacy for more than 6 h.

(2) The combination of sivelestat sodium with morphine can produce a significant synergistic effect in treating or alleviating postoperative pain or posttraumatic pain. This combination not only significantly reduces the dosage of morphine but also further extends the duration of analgesia.

### Embodiment 2

The efficacy of sivelestat sodium in combination with fentanyl or pentazocine was evaluated in a rat model of postoperative pain according to the method in Embodiment 1. For the drug preparation method, reference can be made to Embodiment 1. Fentanyl and pentazocine were prepared into solutions of corresponding concentrations and administered according to the protocol shown in Table 5 below.

TABLE 5
Groups and dosage

| | | Route of | | | Number |
|---|---|---|---|---|---|
| Group | | adminis- | Dosage | Volume | of |
| No. | Group | tration | (mg/kg) | (mL/kg) | animals |
| 12 | Fentanyl | IV | 0.012 | 5 | 8 |
| 13 | Sivelestat | IP + IV | 150 + 0.006 | 5 | 8 |
| | sodium + | | | | |
| | fentanyl | | | | |
| 14 | Pentazocine | SC | 6 | 5 | 8 |
| 15 | Sivelestat | IP + SC | 150 + 6 | 5 | 8 |
| | sodium + | | | | |
| | pentazocine | | | | |

Note:
Route of administration: SC: subcutaneous injection; IP: intraperitoneal injection; and IV: intravenous injection.

The mechanical allodynia threshold (PWT (g)) of each group was measured according to the method in Embodiment 1. The results are shown in Table 6 below.

TABLE 6
Mechanical allodynia threshold (PWT (g)) of each group

| | Mechanical allodynia testing (PWT (g)) | | | | |
|---|---|---|---|---|---|
| | Baseline | | | | |
| Group No. | value | 0.5 h | 1 h | 2 h | 6 h |
| Fentanyl 0.012 mg/kg | 2.8 | 13.6 | 11.9 | 6.6 | 2.9 |
| Sivelestat sodium + | 2.8 | 11.2 | 11.6 | 9.5 | 6.6 |
| fentanyl 150 mg/kg + | | | | | |
| 0.006 mg/kg | | | | | |
| Pentazocine 6 mg/kg | 2.8 | 7.3 | 8.9 | 4.4 | 2.8 |
| Sivelestat sodium + | 2.8 | 7.4 | 9.1 | 6.9 | 4.0 |
| pentazocine | | | | | |
| 150 mg/kg + 6 mg/kg | | | | | |

As can be seen from the results:

The combination of sivelestat sodium with fentanyl can significantly reduce the dosage of fentanyl and prolong the duration of analgesia. The effect of combination of sivelestat sodium with full agonists of μ-opioid receptor (morphine and fentanyl) is significantly superior to that of its combination with the partial/mixed agonist of κ-opioid receptor (pentazocine).

1. A method for treating or alleviating posttraumatic pain or postoperative pain in a subject in need thereof, comprising administering a therapeutically effective amount of sivelestat or a pharmaceutically acceptable salt thereof in combination with an opioid drug.

2. The method according to claim 1, wherein the opioid drug is a full agonist of μ-opioid receptor.

3. The method according to claim 2, wherein the full agonist of μ-opioid receptor comprises morphine, fentanyl, remifentanil, sufentanil, pethidine, oxycodone, and hydromorphone.

4. A combination product comprising the following components:
(1) sivelestat or a pharmaceutically acceptable salt thereof; and
(2) a full agonist of μ-opioid receptor; wherein the full agonist of μ-opioid receptor is selected from morphine, fentanyl, remifentanil, pethidine, oxycodone, and hydromorphone.

5. (canceled)

6. The combination product according to claim 4, wherein the sivelestat or the pharmaceutically acceptable salt thereof is an independent formulation comprising 50-500 mg of the sivelestat or the pharmaceutically acceptable salt thereof, and the full agonist of μ-opioid receptor is an independent formulation comprising 0.01-100 mg of the full agonist of μ-opioid receptor.

7. The combination product according to claim 4, comprising the following components:
(1) sivelestat sodium or a pharmaceutically acceptable salt thereof; and
(2) morphine or fentanyl.

8. A method for treating or alleviating posttraumatic pain or postoperative pain in a subject in need thereof, comprising administering a therapeutically effective amount of the combination product according to claim 4.

9. A pharmaceutical kit comprising the combination product according to claim 4.

10. A method for treating or alleviating posttraumatic pain or postoperative pain in a subject in need thereof, comprising administering a therapeutically effective amount of sivelestat or a pharmaceutically acceptable salt thereof.
